(19) United States
(12) Patent Application Publication
(10) Pub. No.: US 2011/0286979 A1
(43) Pub. Date: Nov. 24, 2011

(54) CHEMOSENSITIZATION BY BI-FUNCTIONAL SMALL HAIRPIN RNA (bi-shRNA)

(75) Inventors: Alex W. Tong, Dallas, TX (US); Yu Yang, Allen, TX (US); Donald Rao, Dallas, TX (US); Phillip B. Maples, Pilot Point, TX (US); Neil Senzer, Dallas, TX (US); John J. Nemunaitis, Cedar Hill, TX (US); Zhaohui Wang, Grapevine, TX (US)

(73) Assignee: GRADALIS, INC., Carrollton, TX (US)

(21) Appl. No.: 13/111,677

(22) Filed: May 19, 2011

Related U.S. Application Data
(60) Provisional application No. 61/346,709, filed on May 20, 2010.

(51) Int. Cl. A61K 35/12; A61K 31/7105; A61K 31/7088; A61P 35/00; A61P 35/04

(52) U.S. Cl. 424/93.21; 514/44 R; 514/44 A

(57) ABSTRACT

Compositions and methods of augmenting the anti-tumor activities of docetaxel and other taxanes by combination with a bi-functional small hairpin RNA (bi-shRNA) is described herein. The instant invention describes the interactive outcome of STMN1 knockdown with docetaxel. In vitro docetaxel (DOC) dose response assessments with or without co-treatment with bi-shRNA^{STMN1} in CCL-247 and SK-MEL-28 melanoma cells indicated that STMN1 knockdown significantly reduced DOC concentration needed to inhibit cancer cell growth by 50% (IC₅₀) of CCL-247 cells from 1.8±0.2 to 0.6±0.4 nm (n=3, p<0.05), and SK-MEL-28 cells from 1.7±0.2 nm to 0.1±0.0 (n=3, p<0.05). The 3- to >10-fold reduction in DOC IC₅₀ suggest that bi-shRNA^{STMN1} can markedly enhance the effectiveness of docetaxel for human cancer cells.

### CROSS-REFERENCE TO RELATED APPLICATIONS

This application claims priority to U.S. Provisional Application Ser. No. 61/346,709, filed May 20, 2010, the contents of which is incorporated by reference herein in its entirety.

### TECHNICAL FIELD OF THE INVENTION

The present invention relates in general to the field of RNA interference (RNAi) treatments for cancers, and more particularly, to the augmentation of the anti-tumor activity of a chemotherapeutic agent in combination with target-specific bi-functional shRNA.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with compositions and methods of augmenting anti-tumor activity of chemotherapeutic agents.

United States Patent Application No. 20100093647 (Liu and Gerson, 2010) discloses compositions and methods useful in the treatment of certain neoplastic disorders based on the recognition that certain molecules that inhibit O6-Methylguanine-DNA-methyltransferase (MGMT) induce, augment, or potentiate mitotic death and the chemotherapeutic efficacy of certain antimitotic agents and DNA damaging agents. The antimitotic agent in the Liu invention are selected from the group consisting of taxol, paclitaxel, docetaxel, and combinations thereof.

United States Patent Application No. 20100028346 (Lutz and Whiteman, 2010) describes a combination of at least one conjugate and one or more chemotherapeutic agent(s) which when administered exerts an unexpectedly enhanced therapeutic effect. The therapeutic effectiveness of the combination is greater than that of the conjugate alone or the administration of one or more of the drug(s) without the conjugate. The Lutz invention is also directed to compositions comprising at least one conjugate and at one or more of chemotherapeutic agent and to methods of treating cancer using at least one conjugate and at least one or more of chemotherapeutic agent(s). The invention also provides methods of modulating the growth of selected cell populations, such as cancer cells, by administering a therapeutically effective amount of one or more chemotherapeutic agent(s) and at least one conjugate. In each case, such combination has therapeutic synergy or improves the therapeutic index in the treatment of cancer over the anticancer agent(s) alone.

### SUMMARY OF THE INVENTION

The instant invention describes the augmentation of docetaxel anti-tumor activity by a stathmin-specific bi-functional shRNA. Post-transcriptional STMN1 knockdown with an expression vector delivered novel bifunctional shRNA (pbi-shRNA^{STMN1}) effectively reduced the growth of multiple human cancer cell types. STMN1 knockdown significantly reduced IC₅₀ of docetaxel from 1.8±0.2 to 0.6±0.4 nm for CCL-247 colorectal cancer cells, and from 1.7±0.2 nm to 0.1±0.0 for SK-Mel-28 melanoma cells. The 3- to >10-fold reduction in DOC IC₅₀ suggests that pbi-shRNA^{STMN1} can be used for chemo-sensitizing human colorectal and melanoma cancer cells to docetaxel. pbi-shRNA^{STMN1} may be used as a biotherapeutic approach in combination with docetaxel.

The anti-mitotic composition for treating one or more cancers as described herein below comprising: one or more chemotherapeutic or anti-tumor agents, wherein the chemotherapeutic agents are selected from the group consisting of taxanes, diterpenes, and other agents acting by mitotic spindle microtubule stabilization and an expression vector comprising: a promoter and a nucleic acid insert operably linked to the promoter, wherein the insert encodes one or more short hairpin RNAs (shRNA) directed against Stathmin 1 (STMN1) and that inhibits the STMN1 protein expression in one or more cancer cells via a RNA interference mechanism. In one aspect the taxanes comprise paclitaxel and docetaxel. In a specific aspect the chemotherapeutic agent is docetaxel used in concentrations ranging from 0.3 nM to 10 nM. In another aspect docetaxel is used in concentrations of 0.3 nM, 0.6 nM, 1.2 nM, 2.5 nM, 5 nM, and 10 nM.

In yet another aspect the one or more cancers are selected from the group consisting of colorectal cancer, breast cancer, melanoma, non-small-cell lung cancer, gall bladder cancer, ovarian, liver cancer, liver cancer metastases, and Ewing's sarcoma. In one aspect the shRNA incorporates one or more siRNA (cleavage-dependent) and miRNA (cleavage-independent) motifs. In another aspect the shRNA is both the cleavage-dependent and cleavage-independent inhibitor of the STMN1 protein expression. In another aspect shRNA is further defined as a bifunctional shRNA. In other aspects the shRNA augments an anti-tumor activity of the one or more chemotherapeutic or anti-tumor agents, wherein the augmentation results in at least 3-fold decrease in an IC₅₀ value of the one or more chemotherapeutic or anti-tumor agents. In a related aspect the one or more short hairpin RNAs (shRNA) are capable of hybridizing to a region of a mRNA transcript encoding furin, PDX-1 oncogene or both thereby inhibiting furin and PDX-1 oncogene expression, respectively via a RNA interference mechanism.

One embodiment of the instant invention provides a method of preventing, treating and/or ameliorating symptoms of a cancer in a patient by comprising the steps of: identifying the patient in need of prevention, treatment, and/or amelioration of the symptoms of the cancer and administering a therapeutically effective amount of an anti-mitotic composition comprising: one or more chemotherapeutic or anti-tumor agents, wherein the chemotherapeutic agents are selected from the group consisting of taxanes, diterpenes, and other agents acting by mitotic spindle microtubule stabilization and an expression vector comprising: a promoter and a nucleic acid insert operably linked to the promoter, wherein the insert encodes one or more short hairpin RNAs (shRNA) directed against Stathmin 1 (STMN1) and that inhibits the STMN1 protein expression in one or more cancer cells via a RNA interference mechanism. The taxanes disclosed comprise paclitaxel and docetaxel, more specifically docetaxel.

In one aspect the docetaxel is used in concentrations ranging from 0.3 nM to 10 nM. In another aspect the docetaxel is used in concentrations of 0.3 nM, 0.6 nM, 1.2 nM, 2.5 nM, 5 nM, and 10 nM. In another aspect the one or more cancers are selected from the group consisting of colorectal cancer, breast cancer, melanoma, non-small-cell lung cancer, gall bladder cancer, ovarian, liver cancer, liver cancer metastases, and Ewing's sarcoma. In one aspect the shRNA incorporates one or more siRNA (cleavage-dependent) and miRNA (cleavage-independent) motifs. In another aspect the shRNA is both the cleavage-dependent and cleavage-independent inhibitor of the STMN1 protein expression. In another aspect the shRNA is further defined as a bifunctional shRNA. In yet another aspect the shRNA augments an anti-tumor activity of the one or more chemotherapeutic or anti-tumor agents, wherein the augmentation results in at least 3-fold decrease in an IC₅₀ value of the one or more chemotherapeutic or anti-tumor agents.

Another embodiment of the present invention describes a composition for treating a colorectal cancer, a breast cancer, a melanoma or a combination thereof comprising: docetaxel or a composition comprising docetaxel with one or more optional pharmaceutically acceptable agents and an expression vector comprising: a promoter and a nucleic acid insert operably linked to the promoter, wherein the insert encodes one or more short hairpin RNAs (shRNA) directed against Stathmin 1 (STMN1) and that inhibits the STMN1 protein expression in one or more cancer cells via a RNA interference mechanism. In one aspect the shRNA incorporates one or more siRNA (cleavage-dependent) and miRNA (cleavage-independent) motifs. In another aspect the shRNA is both the cleavage-dependent and cleavage-independent inhibitor of the STMN1 protein expression. In yet another aspect the shRNA augments an anti-tumor activity of the docetaxel resulting in at least 3-fold decrease in an IC₅₀ value of the docetaxel.

Yet another embodiment of the instant invention deals with a method of preventing, treating and/or ameliorating symptoms of a colorectal cancer, a breast cancer, a melanoma or a combination thereof in a patient by comprising the steps of: (i) identifying the patient in need of prevention, treatment, and/or amelioration of the symptoms of the cancer and (ii) administering a therapeutically effective amount of docetaxel or a composition comprising docetaxel with one or more optional pharmaceutically acceptable agents and an expression vector comprising: a promoter and a nucleic acid insert operably linked to the promoter, wherein the insert encodes one or more short hairpin RNAs (shRNA) directed against Stathmin 1 (STMN1) and that inhibits the STMN1 protein expression in one or more cancer cells via a RNA interference mechanism. In one aspect of the method the shRNA is both the cleavage-dependent and cleavage-independent inhibitor of the STMN1 protein expression. In a specific aspect of the method the shRNA augments an anti-tumor activity of the docetaxel by decreasing the IC₅₀ value by at least 3-fold.

The present invention is also directed towards a method of augmenting the anti-tumor activity of docetaxel or compositions comprising docetaxel comprising the steps of: providing the docetaxel or compositions comprising the docetaxel and adding one or more transfected cancer cells, wherein the cancer cells are transfected with an expression vector comprising: a promoter and a nucleic acid insert operably linked to the promoter, wherein the insert encodes one or more short hairpin RNAs (shRNA) directed against Stathmin 1 (STMN1) and that inhibits the STMN1 protein expression in one or more cancer cells via a RNA interference mechanism. The method describe hereinabove further comprising the step of measuring the augmentation of the anti-tumor activity by a measurement of percent viable cell growth (%) and a viable cell count, wherein a decrease in the percent viable cell growth (%) and the viable cell count in comparison to a control is indicative of the augmentation of anti-tumor activity of docetaxel or compositions comprising the docetaxel. In one aspect the control comprises docetaxel or compositions comprising the docetaxel and one or more cancer cells not transfected with the expression vector. In another aspect the docetaxel is used in concentrations ranging from 0.3 nM to 10 nM. In a related aspect the one or more cancer cells are selected from the group consisting of colorectal cancer cells, breast cancer cells, melanoma cells. In another aspect the shRNA incorporates one or more siRNA (cleavage-dependent) and miRNA (cleavage-independent) motifs. In another aspect the shRNA is both the cleavage-dependent and cleavage-independent inhibitor of the STMN1 protein expression. In yet another aspect the shRNA is further defined as a bifunctional shRNA. In a specific aspects the augmentation results in at least 3-fold decrease in an IC₅₀ value of the docetaxel or compositions comprising docetaxel.

In another aspect the one or more short hairpin RNAs (shRNA) are capable of hybridizing to a region of a mRNA transcript encoding furin, thereby inhibiting furin expression via a RNA interference mechanism. In another aspect the one or more short hairpin RNAs (shRNA) capable of hybridizing to a region of an mRNA transcript that encodes a PDX-1 oncogene and that inhibits the PDX-1 oncogene expression via RNA interference mechanism. The present invention discloses a composition comprising docetaxel augmented by the method described above.

In one embodiment the present invention is an anti-mitotic composition for treating one or more cancers comprising: one or more chemotherapeutic or anti-tumor agents, wherein the chemotherapeutic agents are selected from the group consisting of taxanes, diterpenes, and other agents acting by mitotic spindle microtubule stabilization and an expression vector comprising: a promoter and a nucleic acid insert operably linked to the promoter, wherein the insert encodes one or more bifunctional short hairpin RNAs (shRNA) directed at a target gene, wherein the bifunctional shRNA augments an activity of the one or more chemotherapeutic or anti-tumor agents.

In another embodiment the present invention is directed towards a method of preventing, treating and/or ameliorating symptoms of a cancer in a patient by comprising the steps of: identifying the patient in need of prevention, treatment, and/or amelioration of the symptoms of the cancer and administering a therapeutically effective amount of an anti-mitotic composition comprising: one or more chemotherapeutic or anti-tumor agents, wherein the chemotherapeutic agents are selected from the group consisting of taxanes, diterpenes, and other agents acting by mitotic spindle microtubule stabilization and an expression vector comprising: a promoter and a nucleic acid insert operably linked to the promoter, wherein the insert encodes one or more bifunctional short hairpin RNAs (shRNA) directed at a target gene, wherein the bifunctional shRNA augments an activity of the one or more chemotherapeutic or anti-tumor agents.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B are schematic representation of the concept and construction of pbi-shRNA^{STMN1}: FIG. 1A, FIG. 1B is a circular diagram of expression constructs for shRNA expression. pUMVC3 vector's mammalian expression unit contains enhanced CMV promoter with CMV IE 5′ UTR and partial IE Intron A and rabbit beta-globin poly A site. The shRNA expression unit is inserted in the multiple cloning sites between the CMV IE Intron A and rabbit beta-globin poly A sites;

FIG. 2A shows the STMN1 mRNA target site cleavage detection from the bi-sh-STMN1 transfected CCL-247 cells by 5′ RACE. Photo-image of agarose gel resolving RACE-PCR products. RACE-PCR products were detected in cells transfected with either bi-sh-STMN1 or siRNASTMN1. CCL-247 cells were transfected with 7.22×10-13 M of bi-sh-STMN1 (lane 4), or 30 nM of siRNA (lane 5); a 285 base pairs PCR product was detected (red arrow). Lane 1 is 100 base pair size marker, lane 2 is RNA from un-transfected cells. Lane 3 is RNA from scramble shRNA transfected cells. Lane 6 is PCR only control. Lane 7 is 1 kb size marker;

FIG. 2B is a plot showing STMN1 mRNA knockdown kinetics. SK-MEL-28 cells were reverse-transfected with pGBI-1 (complete matching), or pGBI-2 (bi-functional) or pGBI-3 (with mismatches) at 1 μg/ml concentration. At 24, 48 and 72 hours post-transfection, STMN1 mRNA level were determined by qRT-PCR method normalized to the internal GAPDH mRNA level and percent reduction in STMN1 mRNA were compared with un-transfected cells. pGBI-1 (complete matching, blue line), or pGBI-2 (bi-functional, red line) or pGBI-3 (with mismatches, yellow line);

FIG. 2C shows that bi-sh-STMN1 effectively knocks down STMN1 protein expression in CCL-247 cells. CCL-247 cells were transfected with 3 μg/ml of bi-sh-STMN1. 48 hours after transfection, transfected cells were harvested for immuno-stain with either STMN specific primary antibody (upper panels) or β-Actin specific primary antibody (lower panels). The antibody tagged cells were analyzed by flow cytometry. The result is shown; black line is secondary antibody (phycoerythrin conjugated antibody) fluorescence, the green line is STMN1 specific fluorescence and the red line is β-Actin specific fluorescence;

FIG. 3 is a plot showing the dosing advantage of bi-sh-STMN1 when compared to cleavage-dependent (pGBI-1) and cleavage-independent (pGBI-3) components. Viable cell counts following bi-sh-STMN1 (light blue lines) transfection was compared to the constructs with the single cleavage dependent (pGBI-1, red lines) and independent (pGBI-3, yellow lines) shRNA elements. CCL-247 cells were treated with two doses for each construct; 9.02×10⁻¹⁴ M, or 2.26×10⁻¹⁴ M in comparison to untransfected cells (dark blue lines). Each data point represents a mean of triplicate samples with standard deviation. FIG. 3 shows CCL-247 cells treated with 2.26×10⁻¹⁴ M of constructs; and,

FIGS. 4A and 4B are plots showing the additive anti-tumor activity of bi-shRNA^{STMN}+docetaxel on CCL-247 (human colorectal carcinoma) and SK-Mel-28 (human melanoma) cells, respectively. The plots show BrdU uptake by pbi-shRNA^{STMN1} or pbi-shRNA-scrambled control electroporated (EP) cells at 48 h.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It is contemplated that any embodiment discussed in this specification can be implemented with respect to any method, kit, reagent, or composition of the invention, and vice versa. Furthermore, compositions of the invention can be used to achieve methods of the invention.

The present invention describes the interactive outcome of STMN1 knockdown with docetaxel. The inventors carried out in vitro docetaxel (DOC) dose response assessments with or without co-treatment with bi-shRNA^{STMN1} in CCL-247 and SK-MEL-28 melanoma cells. BrdU determinations indicated that STMN1 knockdown significantly reduced DOC concentration needed to inhibit cancer cell growth by 50% (IC₅₀) of CCL-247 cells from 1.8±0.2 to 0.6±0.4 nm (n=3, p<0.05), and SK-Mel-28 cells from 1.7±0.2 nm to 0.1±0.0 (n=3, p<0.05). The 3- to >10-fold reduction in DOC IC₅₀ suggest that bi-shRNA^{STMN1} can markedly enhance the effectiveness of docetaxel for human cancer cells, and can be potentially used as a biotherapeutic approach in combination with docetaxel.

The inventors have previously developed a bifunctional shRNA construct that mediates stathmin (STMN1) post-transcriptional knockdown through mRNA cleavage-dependent and cleavage-independent mechanisms, and is effective in reducing the growth of human colorectal CCL-247 cells by >80%, correspondingly arresting cycling cells at the G₂M phase. Treatment similarly reduced the growth of human breast cancer (MDA-MB-231) and melanoma (SK-MEL-28) lines by 45% and 48%, respectively. The inventors have previously acknowledged the limitation of RNA interference (RNAi) treatment approaches as monotherapy of advanced cancers and to address this issue they studied the efficacy of bifunctional shRNA in combination with docetaxel chemotherapy in the instant invention. The findings obtained herein show addictive tumor cell growth effect of docetaxel and the bi-functional shRNA construct.

RNA interference (RNAi) is a natural cellular regulatory process capable of inhibiting transcriptional, post-transcriptional and translational mechanisms thereby modulating gene expression¹⁻⁴. RNAi technology is commonly used in reverse genetics approaches to study gene function and to demonstrate targets of therapeutic potential in cancer⁵⁻⁷. Several synthetic methods of silencing gene expression integral to disease phenotype have been developed^{8, 9}. Short hairpin RNA (shRNA) transcribed from an expression vector intrinsically differs from synthetic double stranded small interfering RNA (siRNA) with respect to intracellular trafficking and nucleotide preference¹⁰ and can result in enhanced gene knockdown effects. Recently, the process of RNA interference by endogenously expressed hairpin RNAs, known as microRNAs (miRNAs), has been demonstrated in mammalian cells¹¹. By integrating an siRNA motif in the context of the well known miR30-scaffold, shRNA expressed from constructs of defined specificity against a target gene can be processed via the endogenous miRNA biogenesis pathway¹².

Using a miR30-scaffold, the inventors developed a novel “bifunctional” (bi) RNAi strategy. The inventors hypothesize that a bifunctional construct that concurrently induces translational repression (cleavage-independent mRNA sequestration and degradation) and cleavage-dependent post-transcriptional mRNA degradation can achieve more effective silencing in comparison to siRNA or conventional single-functional shRNA targeted to the same sequence. The bifunctional construct bi-sh-STMN1 is directed against stathmin 1 (STMN1), a gene target candidate that is overexpressed in human cancer lines and was shown by us to be differentially overexpressed in cancer patients, based on mRNA and protein couplet signals in tumor/normal tissue specimen analysis¹³. STMN1 is critically involved in mitotic spindle formation^{14, 15}. Previously studies showed that STMN1 knockdown by conventional siRNA resulted in G₂/M cell cycle arrest, inhibition of clonogenicity, and markedly increased apoptosis¹⁵⁻¹⁷. STMN1 knockdown also produced an additive to synergistic interaction with chemotherapeutic agents such as the taxanes¹⁸⁻²⁰.

The bi-sh-STMN1 incorporates two stem-loop structures in an expression construct promoting both cleavage-dependent and cleavage-independent RNA induced silencing complex (RISC) assemblies thereby generating RISC associated mature effector small RNAs with multiple independent gene silencing activities. Based on the observation that miRNAs are associated with both non-nucleolytic (Ago 1, 3, 4) and nucleolytic Ago2 containing RISC and siRNAs with the nucleolytic Ago2, the novel bifunctional strategy specifically promotes the loading of miRNA-like effector molecules onto the cleavage-independent RISC as well as the accumulation of siRNA effector molecules by the cleavage-dependent (Ago2 containing) RISC²¹. The bifunctional design thermodynamically accommodates passenger strand departure via cleavage dependent and cleavage independent processes so the functionality of the effectors is, thereby, set by programmed passenger strand guided RISC loading rather than being dependent on the Ago protein distribution in the target cell. Insofar as the bifunctional construct uses a natural process (i.e., miRNA biogenesis), the host RNA polymerase II complex can be utilized to allow expression of multiple bifunctional shRNAs targeting multiple key over-expressed genes in tumor with a single primary transcript transcribing from a RNA pol II promoter^{22, 23} structurally analogous to the miR17-92 cluster on chromosome 13 with 6 miRNAs expressed in a poly-cistronic fashion²⁴.

The construction of STMN1 uni-functional shRNA (pGBI-1, pGBI-3) and bi-functional shRNA (bi-sh-STMN1, or pGBI-2) has been previously described by the inventors.

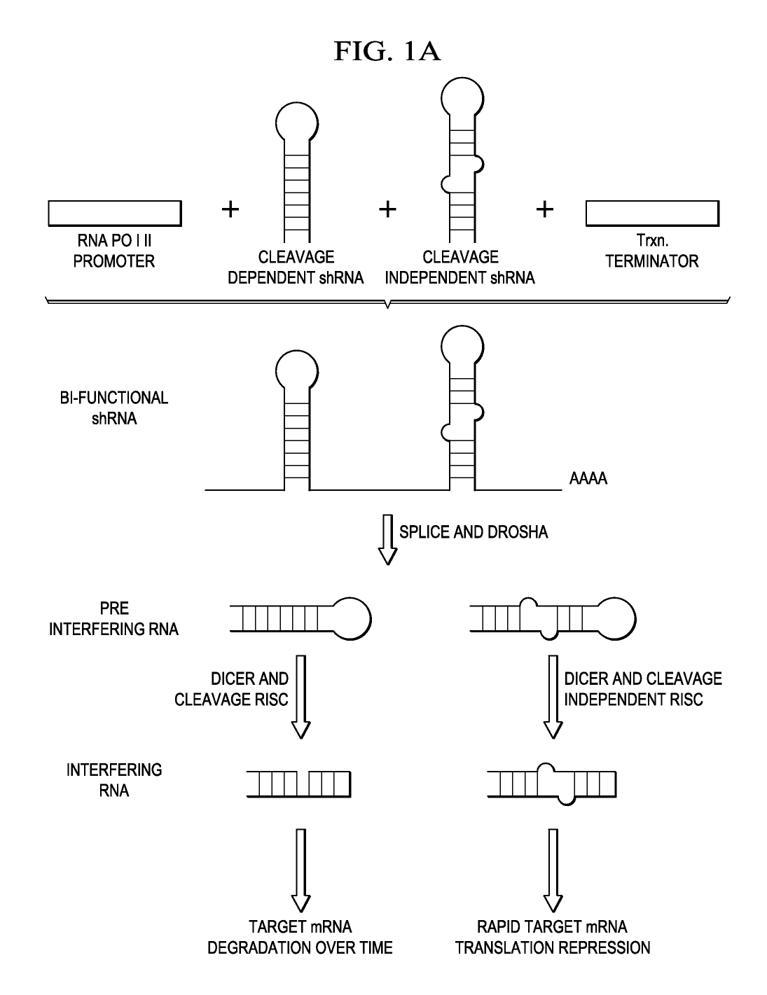

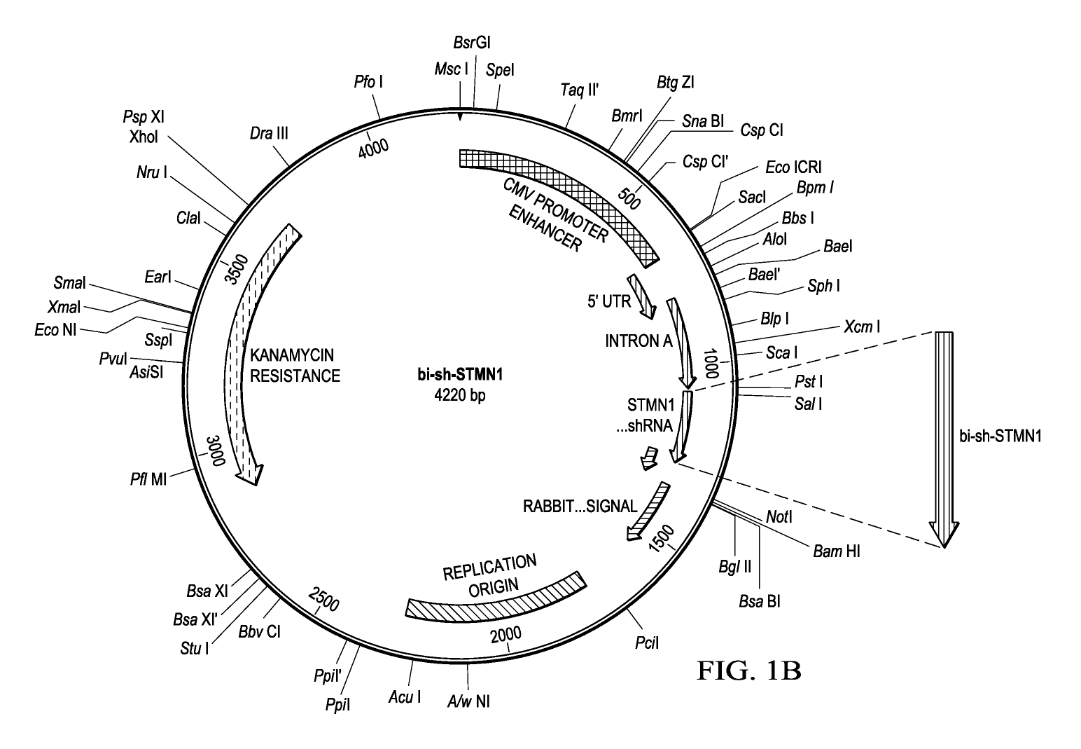

The enhanced shRNAs employed in the present invention comprise both types of shRNAs, namely, shRNAs designed to enter into and interact with both cleavage-dependent RISC and cleavage-independent RISC (FIG. 1A). The invention provides that a higher level of gene “knock-down,” i.e., translation repression of Target Gene mRNA transcripts, is achieved using such enhanced shRNAs than other currently-available RNAi methods and compositions.

More specifically, the present invention provides methods and compositions for the synthesis of novel shRNA molecules that may be transcribed endogenously in human, animal and plant cells, for the purpose of “knocking down” the expression of one or more Targeted Genes. The shRNAs of the present invention simultaneously enter both cleavage-dependent RISCs and cleavage-independent RISCs, and inhibit the expression of a targeted mRNA containing a complementary target sequence (FIG. 1A).

The expression unit for the bifunctional shRNA to Stathmin1 (bi-sh-STMN1) was inserted between the Sal I and Not I sites of mammalian expression vector pUMVC3 (FIG. 1B) and is driven from an enhanced [pol II] CMV promoter²⁵. It contains two stem-loop structures, one with complete matching passenger and guide strands (cleavage-dependent), and the other with two base-pair mismatches between passenger and guide strands (cleavage-independent). The GC to AU switches are at positions 11 and 12 of the passenger strand which create mismatches at the central location similar to most miRNAs 26 (Predicted secondary structure shown in Table I).

TABLE I
shRNA sequences inserted into the multiple cloning sites of pUMVC3 for bi-sh-
STMN1 (pGBI-2), pGBI-1 (cleavage dependent component) and pGBI-3 (cleavage independent
component).

| Plasmids | shRNA Sequence | Predicted Mechanism |
|---|---|---|
| pGBI-1 | 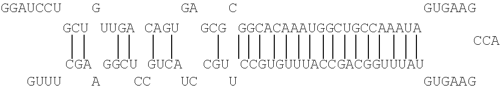 | Cleavage Dependent |
| bi-sh- STMN1 | 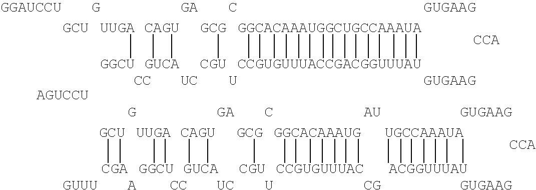 | Bi-functional |
| pGBI-3 | 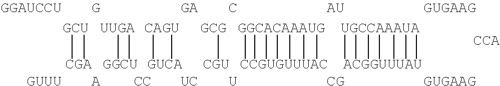 | Cleavage Independent |

The STMN1 mRNA target site we selected was based on maximal knockdown efficacy as determined by comparison of several commercially available siRNA^{STMN1}s. The selected target site was also screened with the BLAST local alignment program to limit the potential matches or “seed sequence” matches with other human transcripts. For comparative purposes and for consistency, the selected STMN1 mRNA target site was used for siRNA and for all shRNA expression constructs throughout the studies describe herein. To test whether the STMN1 expression could be effectively knocked down with each of the separate components of the bi-sh-STMN1 construct, CCL-247 cells were transfected with either 1, 2 or 3 μg/ml of the pGBI-1 (cleavage-dependent component) or of the pGBI-3 (cleavage-independent component) plasmid. Both effectors at all three doses knocked down STMN1 protein expression at 48 hours post-transfection (data not shown).

For in vitro studies, the inventors chose to demonstrate activity in cell lines over-expressive of STMN1 protein. The inventors compared a battery of cell lines and found a wide variation of STMN1 protein expression which could be grouped into low, medium and high STMN1 expressing cells by normalizing to STMN1 expression in peripheral blood cells (PBC, with lowest STMN1 expression); low STMN1 expressing cells are less than 5 fold elevated from PBC's, medium expressing cells are less than 20 fold elevated, while high expressing cells are more than 20 fold elevated in comparative expression (data not shown). Two medium STMN1 expressing cell lines, colon cancer cell line CCL-247 and melanoma cell line SK-MEL-28, and a high STMN1 expressing cell line, breast cancer cell line MDA-MB-231 were selected for the in vitro studies.

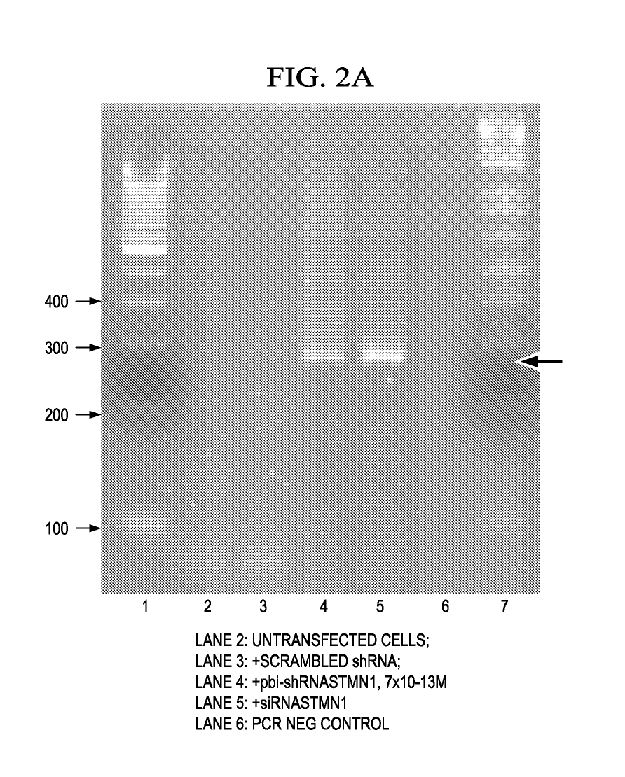

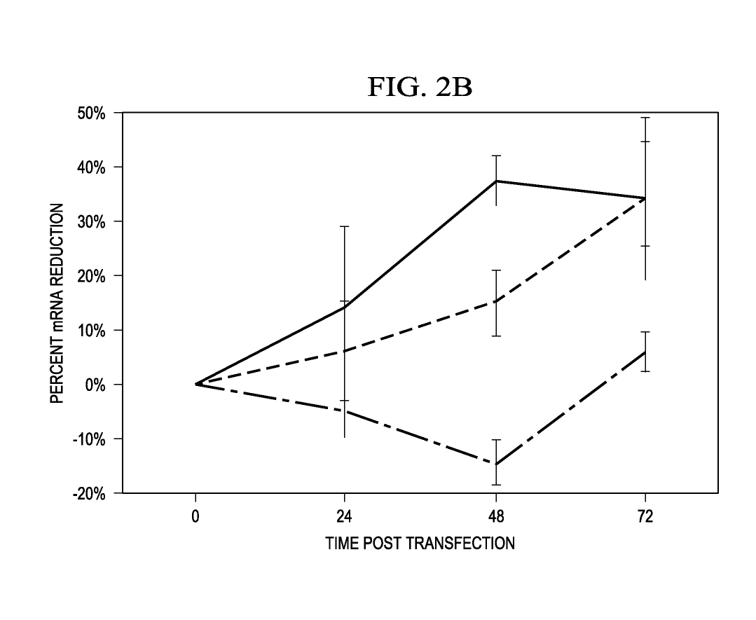

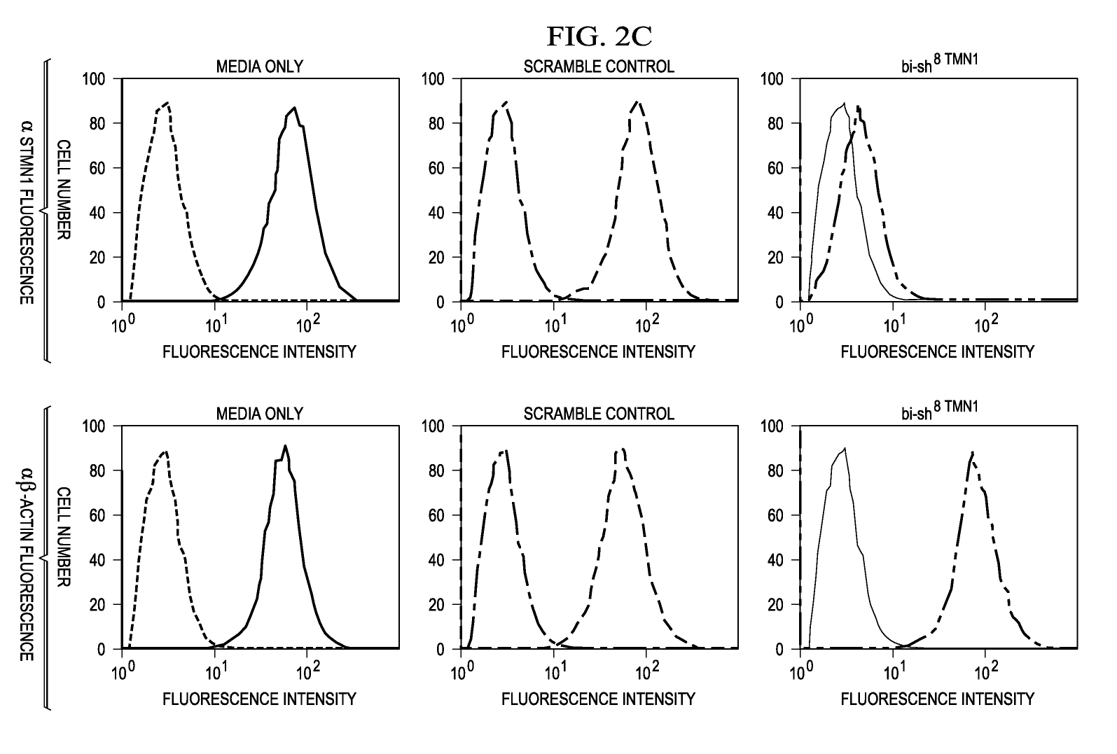

To validate the target cleavage sites as predicted from the cleavage-dependent siRNA component of bi-sh-STMN1²⁶ the 5′ Rapid Amplification of cDNA Ends (5′ RACE) method was used. The inventors designed gene specific primers both for reverse transcription (RT) and for PCR. For PCR, the inventors also used the gene specific nested primer strategy to reduce any non-specific background. The RACE PCR product with predicted size was detected in cells transfected with either bi-sh-STMN1 or siRNASTMN1 (FIG. 2A, lanes 4 or 5, respectively, red arrow), and was confirmed by DNA sequencing to represent a STMN1 mRNA fragment following cleavage between nucleotide position 10 and 11 of the sense strand.

To demonstrate the specificity of target protein knockdown, following transfection with bi-sh-STMN1, CCL-247 cells were tagged with STMN1 specific antibody for flow cytometry analysis (FCA). At 48 hours post transfection, FCA demonstrated 93% reduction in STMN1 protein expression (shift in fluorescent intensity, FIG. 2C, upper panels) as compared with untreated control. By contrast, treatment with the scramble control did not result in STMN1 reduction. β-actin expression was not changed by bi-sh-STMN1 treatment (FIG. 2C, lower panels).

The inventors also evaluated the mRNA knockdown kinetics of the composite bifunctional construct (pGBI-2) compared to constructs comprised of each of the individual components using SK-MEL-28 cells. FIG. 2B illustrates the comparative target mRNA knockdown kinetics over the 72 hours post-exposure to pGBI-1, -3 and bi-sh-STMN1 (pBGI-2) as determined by qRT-PCR. The qRT-PCR primers were designed to flank the target site for the most effective detection of target site cleavage mediated STMN1 mRNA knockdown. pGBI-1 (cleavage-dependent mRNA degradation only) induced STMN1 mRNA knockdown was evident at 24 hours, peaking at 48 hours. By comparison, with pGBI-3 treatment (via translational inhibition and/or sequestration in the p-body with or without subsequent mRNA deadenylation, decapping, and degradation, despite the same target sequence)^{27, 28} STMN1 mRNA was more abundant at 24 and 48 hours compared to the un-treated cells and started to decline only at 72 hours post-transfection. The STMN1 mRNA knockdown response to the bi-sh-STMN1 (pGBI-2) was evident at 48 hours and continued to increase at 72 hours. We could not ascertain changes at later time points due to the limitations of the in vitro transfection system (untransfected cell growth).

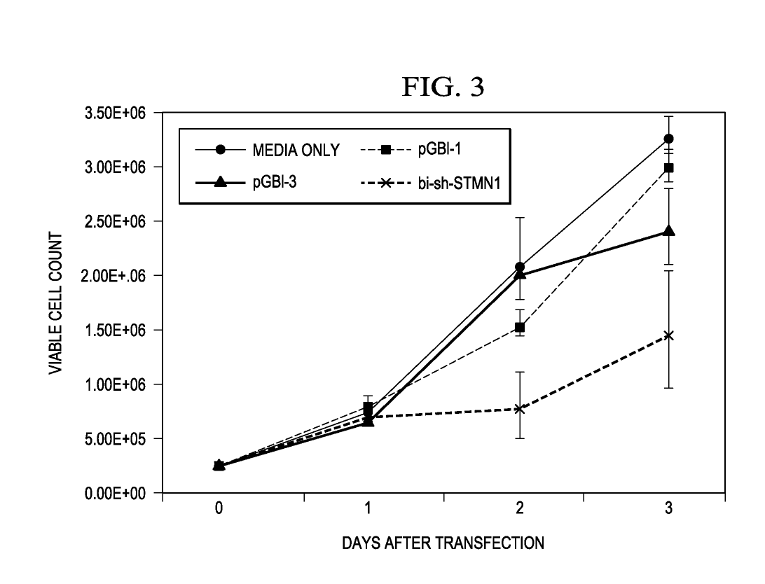

The inventors then examined the bi-sh-STMN1 construct's effect on cancer cell growth inhibition over 72 hours as compared to each of it's individual components (pGBI-1 and pGBI-3) at three different doses that are lower than the IC50 for the bifunctional with 3.61×10⁻¹³ M as the highest dose and two additional lower doses (9.02×10⁻¹⁴ M and 2.26×10⁻¹⁴ M). Reverse transfection of CCL-247 cells was performed with the three different concentrations for each construct. At the highest concentration (3.61×10⁻¹³ M), all three constructs inhibited cancer cell growth equally (data not shown). At the 9.02×10⁻¹⁴ M, all three constructs were observed to significantly inhibit CCL-247 growth for the three day period when compared to the no treatment control (data not shown), but the bi-sh-STMN1 construct was able to sustain growth inhibition more effectively through day 3 when compared to the single component construct (pGBI-2 vs. pGBI-1, p=0.002; pGBI-2 vs. pGBI-3, p=0.003). Even at the lowest dose (2.26×10⁻¹⁴ M), bi-sh-STMN1 demonstrated a significant difference in growth inhibition compared to both the individual cleavage-dependent (pGBI-1) and -independent (pGBI-3) constructs (bi-sh-STMN1 vs. pGBI-1, p<0.001, bi-sh-STMN1 vs. pGBI-3, p<0.001) (FIG. 3).

The present inventors studied the combination cancer growth inhibition effect of anti-stathmin shRNA and chemotherapeutic agent docetaxel on human colon adenocarcinoma CCL-247 cells herein. Docetaxel is one of the chemotherapeutic agents in the taxane group. Previous studies have indicated that taxane stabilize microtubules of the mitotic spindle. To further enhance the potency of shRNA interference therapy, the inventors studied additional/synergistic cell growth inhibition effect with docetaxel. The details of these studies are described in detail herein below:

Docetaxel storage stock was made in 10 mM, with DMSO as solvent in a biological safety cabinet for sterility purposes. Docetaxel solution was stored in 4° C. fridge away from direct light. For each experiment, docetaxel storage solution was brought to room temperature to thaw, about 30 min before usage. A 5 uM working solution was made and further diluted 1:50. To 9.8 ml complete CCL-247 media, 0.2 ml 5 uM working solution was added so that 10 ul of the solution into final volume of 100 ul will make 10 nM. The 10 nM solution was serially diluted to make 5 nM, 2.5 nM, 1.25 nM, 0.625 nM, and 0.3124 nM.

The CCL-247 media comprises HyClone McCoy's 5A medium 500 ml, FBS 50 ml, and L-Glutamine 10 ml. Under the biological cabinet, the above solutions were mixed into McCoy's 5A medium 500 ml bottle. The mixture was poured at the top part of filter, a vacuum pump was turned on to filter the mixed medium through. The media was stored at 4° C. after use.

Cells are cultured and grown to sufficient amount for the studies. The shRNA plasmid transfection was performed by electroporation, in the dose of 50 ug/10 million cell. Finally the different ingredients were placed in 96-well plates (media, transfected cells with and without docetaxel, buffer) and incubated at 37° C. with 5% CO₂. Growth inhibition was studied using the BrdU Cell Proliferation Assay kit at 24, 48, and 72 hrs.

The cell growth inhibition for each of treatment is calculated from direct OD reading. The % growth inhibition is calculated as:

% growth inhibition of control=OD_{treated}−OD_{treatedNoBrdUcontrol}/OD_{untreated}−OD_{untreatedNoBrdUcontrol}

wherein, OD_{treated}: OD reading from the shRNA plasmid and/or docetaxel treated reactant; OD_{treatedNoBrdUcontrol}: OD reading from the shRNA plasmid and/or docetaxel treated reactant, but without BrdU labeling; OD_{untreated}: OD reading from reactants without shRNA plasmid or docetaxel treatment; OD_{untreatedNoBrdUcontrol}: OD reading from reactants without shRNA plasmid or docetaxel treatment, and without BrdU labeling.

The % growth inhibition is calculated and plotted against treatment dose of docetaxel. From the plotted % growth inhibition IC₅₀ is determined from curve for shRNA with/without docetaxel, as well as with docetaxel in different concentrations. IC₅₀—half maximal inhibitory concentration, is a measure of the effectiveness of a compound (docetaxel for this study) in inhibiting biological function (cell growth for this study).

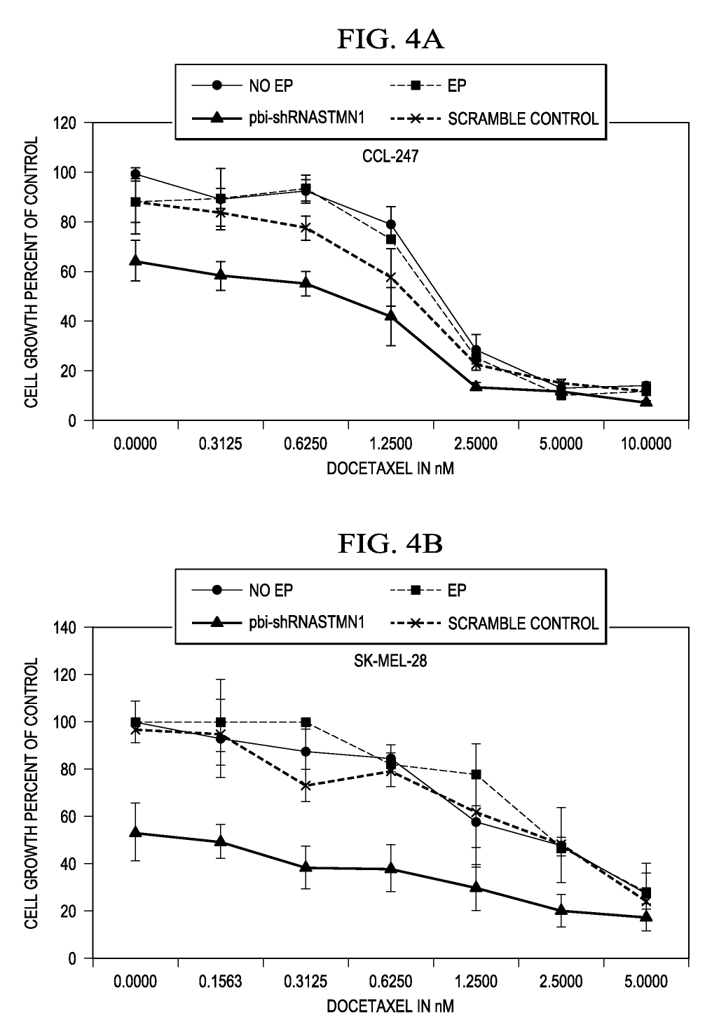

The plots showing the findings of the study are presented in FIGS. 4A and 4B. Table II shows increased docetaxel sensitivity following bi-shRNA^{STMN1} (pGBI-2) transfection.

TABLE II
Increased docetaxel sensitivity following bi-shRNA^{STMN1}
(pGBI-2) transfection.

| | Docetaxel IC50 (nM; mean ± SD) | | | | | |
|---|---|---|---|---|---|---|
| | CCL-2471 | | | SK-Mel-281 | | |
| | | + | + | | + | + |
| | no | pbi-sh- | scrambled | no | pbi-sh- | scrambled |
| Time | ShRNA | RNA^{STMN1} | control | ShRNA | RNA^{STMN1} | control |
| 48 h | 1.8 ± 0.2 | 0.6 ± 0.4** | 1 3 ± 0.3 | 1.7 ± 02 | 0.1 ± 0.0** | 1.0 ± 0.2* |
| 72 h | 1.0 ± 0.4 | 0.6 ± 0.4 | 1.0 ± 0.6 | 0.8 ± 0.2 | 0.2 ± 0.1* | 0.9 ± 0.2 |

pbi-shRNA^{STMN1} co-treatment significantly reduced docetaxel IC₅₀, particularly at 48 h post-treatment (*, p < 0.05; **, p < 0.01; one way ANOVA). pbi-shRNA^{STMN1} may have a more profound docetaxel-augmenting effect on SK-Mel-28 melanoma cells (All cells underwent electroporation. Electroporation alone did not significantly alter IC50 for both cell lines).

Cell growth data at 48 hrs showed: docetaxel alone in the concentration of 1.25 nM had about 70% cell growth compared to control; however, the docetaxel combined with pGBI-2 shRNA had about 35% cell growth compared to control. Considering pGBI-2 shRNA caused about 40% of cell growth inhibition (because it showed 60% cell growth compared to control), the combination effect of docetaxel to pGBI-2 are additive. STMN1 knockdown significantly reduced IC₅₀ of docetaxel from 1.8±0.2 to 0.6±0.4 nm for CCL-247 colorectal cancer cells. The 3- to >10-fold reduction in DOC IC₅₀ suggests that pbi-shRNASTMN1 can be used for chemosensitizing human colorectal cancer cells to docetaxel.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

United States Patent Application No. 20100093647: MGMT inhibitor combination for the treatment of neoplastic disorders.
United States Patent Application No. 20100093647: Novel synergistic effects.
1. Irvine D V, Zaratiegui M, Tolia N H, Goto D B, Chitwood D H, Vaughn M W et al. Argonaute slicing is required for heterochromatic silencing and spreading. Science 2006; 313(5790): 1134-1137.
2. Bailis J M, Forsburg S L. RNAi hushes heterochromatin. Genome biology 2002; 3(12): REVIEWS 1035.
3. Chu C Y, Rana T M. Small RNAs: regulators and guardians of the genome. Journal of cellular physiology 2007; 213(2): 412-419.
4. Ghildiyal M, Zamore P D. Small silencing RNAs: an expanding universe. Nature reviews 2009; 10(2): 94-108.
5. Grimm D, Kay M A. Therapeutic application of RNAi: is mRNA targeting finally ready for prime time? The Journal of clinical investigation 2007; 117(12): 3633-3641.
6. Gewirtz A M. On future's doorstep: RNA interference and the pharmacopeia of tomorrow. The Journal of clinical investigation 2007; 117(12): 3612-3614.
7. Silva J, Chang K, Hannon G J, Rivas F V. RNA-interference-based functional genomics in mammalian cells: reverse genetics coming of age. Oncogene 2004; 23(51): 8401-8409.
8. Wang S, Shi Z, Liu W, Jules J, Feng X. Development and validation of vectors containing multiple siRNA expression cassettes for maximizing the efficiency of gene silencing. BMC Biotechnol 2006; 6: 50.
9. Liu Y P, Haasnoot J, ter Brake 0, Berkhout B, Konstantinova P. Inhibition of HIV-1 by multiple siRNAs expressed from a single microRNA polycistron. Nucleic acids research 2008; 36(9): 2811-2824.
10. Li L, Lin X, Khvorova A, Fesik S W, Shen Y. Defining the optimal parameters for hairpin-based knockdown constructs. RNA (New York, N.Y. 2007; 13(10): 1765-1774.
11. Pillai R S. MicroRNA function: multiple mechanisms for a tiny RNA? RNA (New York, N.Y. 2005; 11(12): 1753-1761.
12. Zeng Y, Cai X, Cullen B R. Use of RNA polymerase II to transcribe artificial microRNAs. Methods Enzymol 2005; 392: 371-380.
13. Nemunaitis J, Senzer N, Khalil I, Shen Y, Kumar P, Tong A et al. Proof concept for clinical justification of network mapping for personalized cancer therapeutics. Cancer gene therapy 2007; 14(8): 686-695.
14. Rana S, Maples P B, Senzer N, Nemunaitis J. Stathmin 1: a novel therapeutic target for anticancer activity. Expert review of anticancer therapy 2008; 8(9): 1461-1470.
15. Iancu C, Mistry S J, Arkin S, Wallenstein S, Atweh G F. Effects of stathmin inhibition on the mitotic spindle. Journal of cell science 2001; 114(Pt 5): 909-916.
16. Alli E, Yang J M, Hait W N. Silencing of stathmin induces tumor-suppressor function in breast cancer cell lines harboring mutant p53. Oncogene 2006.
17. Zhang H Z, Wang Y, Gao P, Lin F, Liu L, Yu B et al. Silencing Stathmin Gene Expression by Survivin Promoter-Driven siRNA Vector to Reverse Malignant Phenotype of Tumor Cells. Cancer biology & therapy 2006; 5(11): 1457-1461.
18. Mistry S J, Atweh G F. Therapeutic interactions between stathmin inhibition and chemotherapeutic agents in prostate cancer. Molecular cancer therapeutics 2006; 5(12): 3248-3257.
19. Ngo T T, Peng T, Liang X J, Akeju O, Pastorino S, Zhang W et al. The 1p-encoded protein stathmin and resistance of malignant gliomas to nitrosoureas. Journal of the National Cancer Institute 2007; 99(8): 639-652.
20. Wang R, Dong K, Lin F, Wang X, Gao P, Wei S H et al Inhibiting proliferation and enhancing chemosensitivity to taxanes in osteosarcoma cells by RNA interference-mediated downregulation of stathmin expression. Molecular medicine (Cambridge, Mass. 2007; 13(11-12): 567-575.
21. Azuma-Mukai A, Oguri H, Mituyama T, Qian Z R, Asai K, Siomi H et al. Characterization of endogenous human Argonautes and their miRNA partners in RNA silencing. Proceedings of the National Academy of Sciences of the United States of America 2008; 105(23): 7964-7969.
22. Lee Y, Kim M, Han J, Yeom K H, Lee S, Baek S H et al. MicroRNA genes are transcribed by RNA polymerase II. The EMBO journal 2004; 23(20): 4051-4060.
23. Lagos-Quintana M, Rauhut R, Meyer J, Borkhardt A, Tuschl T. New microRNAs from mouse and human. RNA (New York, N.Y. 2003; 9(2): 175-179.
24. Mendell J T. miRiad roles for the miR-17-92 cluster in development and disease. Cell 2008; 133(2): 217-222.
25. Pizzorno M C, O'Hare P, Sha L, LaFemina R L, Hayward G S. trans-activation and autoregulation of gene expression by the immediate-early region 2 gene products of human cytomegalovirus. Journal of virology 1988; 62(4): 1167-1179.
26. Soutschek J, Akinc A, Bramlage B, Charisse K, Constien R, Donoghue M et al. Therapeutic silencing of an endogenous gene by systemic administration of modified siRNAs. Nature 2004; 432(7014): 173-178.
27. Roush S F, Slack F J. Micromanagement: a role for microRNAs in mRNA stability. ACS chemical biology 2006; 1(3): 132-134.
28. Wu L, Fan J, Belasco J G. MicroRNAs direct rapid deadenylation of mRNA. Proceedings of the National Academy of Sciences of the United States of America 2006; 103(11): 4034-4039.

What is claimed is:

1. An anti-mitotic composition for treating one or more cancers comprising:
one or more chemotherapeutic or anti-tumor agents, wherein the chemotherapeutic agents are selected from the group consisting of taxanes, diterpenes, and other agents acting by mitotic spindle microtubule stabilization; and
an expression vector comprising: a promoter and a nucleic acid insert operably linked to the promoter, wherein the insert encodes one or more short hairpin RNAs (shRNA) directed against Stathmin 1 (STMN1) and that inhibits the STMN1 protein expression in one or more cancer cells via a RNA interference mechanism.

2. The composition of claim 1, wherein the taxanes comprise paclitaxel and docetaxel.

3. The composition of claim 1, wherein the chemotherapeutic agent is docetaxel.

4. The composition of claim 4, wherein the docetaxel is used in concentrations ranging from 0.3 nM to 10 nM.

5. The composition of claim 4, wherein the docetaxel is used in concentrations of 0.3 nM, 0.6 nM, 1.2 nM, 2.5 nM, 5 nM, and 10 nM.

6. The composition of claim 1, wherein the one or more cancers are selected from the group consisting of colorectal cancer, breast cancer, melanoma, non-small-cell lung cancer, gall bladder cancer, ovarian, liver cancer, liver cancer metastases, and Ewing's sarcoma.

7. The composition of claim 1, wherein the shRNA incorporates one or more siRNA (cleavage-dependent) and miRNA (cleavage-independent) motifs.

8. The composition of claim 1, wherein the shRNA is both the cleavage-dependent and cleavage-independent inhibitor of the STMN1 protein expression.

9. The composition of claim 1, wherein the shRNA is further defined as a bifunctional shRNA.

10. The composition of claim 1, wherein the shRNA augments an anti-tumor activity of the one or more chemotherapeutic or anti-tumor agents.

11. The composition of claim 10, wherein the augmentation results in at least 3-fold decrease in an IC₅₀ value of the one or more chemotherapeutic or anti-tumor agents.

12. The composition of claim 1, wherein the one or more short hairpin RNAs (shRNA) are capable of hybridizing to a region of a mRNA transcript encoding furin, thereby inhibiting furin expression via a RNA interference mechanism.

13. The composition of claim 1, wherein the one or more short hairpin RNAs (shRNA) capable of hybridizing to a region of an mRNA transcript that encodes a PDX-1 oncogene and that inhibits the PDX-1 oncogene expression via RNA interference mechanism.

14. A method of preventing, treating and/or ameliorating symptoms of a cancer in a patient by comprising the steps of:
identifying the patient in need of prevention, treatment, and/or amelioration of the symptoms of the cancer; and
administering a therapeutically effective amount of an anti-mitotic composition comprising: one or more chemotherapeutic or anti-tumor agents, wherein the chemotherapeutic agents are selected from the group consisting of taxanes, diterpenes, and other agents acting by mitotic spindle microtubule stabilization and an expression vector comprising: a promoter and a nucleic acid insert operably linked to the promoter, wherein the insert encodes one or more short hairpin RNAs (shRNA) directed against Stathmin 1 (STMN1) and that inhibits the STMN1 protein expression in one or more cancer cells via a RNA interference mechanism.

15. The method of claim 14, wherein the taxanes comprise paclitaxel and docetaxel.

16. The method of claim 14, wherein the chemotherapeutic agent is docetaxel.

17. The method of claim 16, wherein the docetaxel is used in concentrations ranging from 0.3 nM to 10 nM.

18. The method of claim 16, wherein the docetaxel is used in concentrations of 0.3 nM, 0.6 nM, 1.2 nM, 2.5 nM, 5 nM, and 10 nM.

19. The method of claim 14, wherein the one or more cancers are selected from the group consisting of colorectal cancer, breast cancer, melanoma, non-small-cell lung cancer, gall bladder cancer, ovarian, liver cancer, liver cancer metastases, and Ewing's sarcoma.

20. The method of claim 14, wherein the shRNA incorporates one or more siRNA (cleavage-dependent) and miRNA (cleavage-independent) motifs.

21. The method of claim 14, wherein the shRNA is both the cleavage-dependent and cleavage-independent inhibitor of the STMN1 protein expression.

22. The method of claim 14, wherein the shRNA is further defined as a bifunctional shRNA.

23. The method of claim 14, wherein the shRNA augments an anti-tumor activity of the one or more chemotherapeutic or anti-tumor agents.

24. The method of claim 23, wherein the augmentation results in at least 3-fold decrease in an IC₅₀ value of the one or more chemotherapeutic or anti-tumor agents.

25. A composition for treating a colorectal cancer, a breast cancer, a melanoma or a combination thereof comprising:
docetaxel or a composition comprising docetaxel with one or more optional pharmaceutically acceptable agents; and
an expression vector comprising: a promoter and a nucleic acid insert operably linked to the promoter, wherein the insert encodes one or more short hairpin RNAs (shRNA) directed against Stathmin 1 (STMN1) and that inhibits the STMN1 protein expression in one or more cancer cells via a RNA interference mechanism.

26. The composition of claim 25, wherein the shRNA incorporates one or more siRNA (cleavage-dependent) and miRNA (cleavage-independent) motifs.

27. The composition of claim 25, wherein the shRNA is both the cleavage-dependent and cleavage-independent inhibitor of the STMN1 protein expression.

28. The composition of claim 25, wherein the shRNA augments an anti-tumor activity of the docetaxel.

29. The composition of claim 28, wherein the augmentation results in at least 3-fold decrease in an IC₅₀ value of the docetaxel.

30. A method of preventing, treating and/or ameliorating symptoms of a colorectal cancer, a breast cancer, a melanoma or a combination thereof in a patient by comprising the steps of:
identifying the patient in need of prevention, treatment, and/or amelioration of the symptoms of the cancer; and
administering a therapeutically effective amount of docetaxel or a composition comprising docetaxel with one or more optional pharmaceutically acceptable agents and an expression vector comprising: a promoter and a nucleic acid insert operably linked to the promoter, wherein the insert encodes one or more short hairpin RNAs (shRNA) directed against Stathmin 1 (STMN1) and that inhibits the STMN1 protein expression in one or more cancer cells via a RNA interference mechanism.

31. The method of claim 30, wherein the shRNA is both the cleavage-dependent and cleavage-independent inhibitor of the STMN1 protein expression.

32. The method of claim 30, wherein the shRNA augments an anti-tumor activity of the docetaxel.

33. The method of claim 32, wherein the augmentation results in at least 3-fold decrease in an IC₅₀ value of the docetaxel.

34. A method of augmenting the anti-tumor activity of docetaxel or compositions comprising docetaxel comprising the steps of:
providing the docetaxel or compositions comprising the docetaxel; and
adding one or more transfected cancer cells, wherein the cancer cells are transfected with an expression vector comprising: a promoter and a nucleic acid insert operably linked to the promoter, wherein the insert encodes one or more short hairpin RNAs (shRNA) directed against Stathmin 1 (STMN1) and that inhibits the STMN1 protein expression in one or more cancer cells via a RNA interference mechanism.

35. The method of claim 34, further comprising the step of measuring the augmentation of the anti-tumor activity by a measurement of percent viable cell growth (%) and a viable cell count, wherein a decrease in the percent viable cell growth (%) and the viable cell count in comparison to a control is indicative of the augmentation of anti-tumor activity of docetaxel or compositions comprising the docetaxel.

36. The method of claim 35, wherein the control comprises docetaxel or compositions comprising the docetaxel and one or more cancer cells not transfected with the expression vector.

37. The method of claim 34, wherein the docetaxel is used in concentrations ranging from 0.3 nM to 10 nM.

38. The method of claim 34, wherein the one or more cancer cells are selected from the group consisting of colorectal cancer cells, breast cancer cells, melanoma cells.

39. The method of claim 34, wherein the shRNA incorporates one or more siRNA (cleavage-dependent) and miRNA (cleavage-independent) motifs.

40. The method of claim 34, wherein the shRNA is both the cleavage-dependent and cleavage-independent inhibitor of the STMN1 protein expression.

41. The method of claim 34, wherein the shRNA is further defined as a bifunctional shRNA.

42. The method of claim 34, wherein the augmentation results in at least 3-fold decrease in an IC₅₀ value of the docetaxel or compositions comprising docetaxel.

43. The method of claim 34, wherein the one or more short hairpin RNAs (shRNA) are capable of hybridizing to a region of a mRNA transcript encoding furin, thereby inhibiting furin expression via a RNA interference mechanism.

44. The method of claim 34, wherein the one or more short hairpin RNAs (shRNA) capable of hybridizing to a region of an mRNA transcript that encodes a PDX-1 oncogene and that inhibits the PDX-1 oncogene expression via RNA interference mechanism.

45. A composition comprising docetaxel augmented by the method of claim 34.

46. An anti-mitotic composition for treating one or more cancers comprising:
one or more chemotherapeutic or anti-tumor agents, wherein the chemotherapeutic agents are selected from the group consisting of taxanes, diterpenes, and other agents acting by mitotic spindle microtubule stabilization; and
an expression vector comprising: a promoter and a nucleic acid insert operably linked to the promoter, wherein the insert encodes one or more bifunctional short hairpin RNAs (shRNA) directed at a target gene, wherein the bifunctional shRNA augments an activity of the one or more chemotherapeutic or anti-tumor agents.

47. A method of preventing, treating and/or ameliorating symptoms of a cancer in a patient by comprising the steps of:
identifying the patient in need of prevention, treatment, and/or amelioration of the symptoms of the cancer; and
administering a therapeutically effective amount of an anti-mitotic composition comprising: one or more chemotherapeutic or anti-tumor agents, wherein the chemotherapeutic agents are selected from the group consisting of taxanes, diterpenes, and other agents acting by mitotic spindle microtubule stabilization and an expression vector comprising: a promoter and a nucleic acid insert operably linked to the promoter, wherein the insert encodes one or more bifunctional short hairpin RNAs (shRNA) directed at a target gene, wherein the bifunctional shRNA augments an activity of the one or more chemotherapeutic or anti-tumor agents.
